SEQUENCE LISTING (1 sequence)

SEQ ID NO: 1 - protein, 245 aa, Homo sapiens
  CHAIN (208)..(245): Sequence entails the binding epitope of the monoclonal antibody 48D19
DEPMDFKINTDEIMTSLKSVNGQIESLISPDGSRKNPARNCRDLKFCHPELKSGEYWVDPNQGCKLDAIKVFCNMETGETCISANPLNVPRKHWWTDSSAEKKHVWFGESMDGGFQFSYGNPELPEDVLDVQLAFLRLLSSRASQNITYHCKNSIAYMDQASGNVKKALKLMGSNEGEFKAEGNSKFTYTVLEDGCTKHTGEWSKTVFEYRTRKAVRLPIVDIAPYDIGGPDQEFGVDVGPVCFL

(19) United States
(12) Patent Application Publication
(10) Pub. No.: US 2005/0074820 A1
(43) Pub. Date: Apr. 7, 2005

(54) Method for the immunohistological detection of fibrogenesis in histological tissue sections using antiboides against C-terminal procollagen alpha1 (III) propeptide

(76) Inventors: Elmar Reinhold Burchardt, Wuppertal (DE); David A. Brenner, Chapel Hill, NC (US)

Correspondence Address: ELMAR R. BURCHARDT, DAMASCHREWEG 69, WUPPERTAL, 42113, DE

(21) Appl. No.: 10/820,773

(22) Filed: Apr. 9, 2004

Related U.S. Application Data
(63) Continuation of application No. PCT/DE02/03763, filed on Oct. 6, 2002.

(30) Foreign Application Priority Data
Oct. 6, 2001 (DE) 101 49 360.6
Oct. 6, 2001 (WO) 03/034070 A2

(51) Int. Cl. G01N 33/53; G01N 33/567

(52) U.S. Cl. 435/7.2; 514/2

(57) ABSTRACT

The invention relates to a method for specific coloration of collagen-producing cells in tissue cuts for immunohistological diagnosis of fibrotic diseases using antibodies against human C-terminal procollagen α₁ (III) propeptide (PIIICP).

This application is a continuation of International Application PCT/DE02/03763 with an international filing date of Oct. 6^{th}, 2002, not published in the English language under PCT Article 21 (2), and now abandoned.

The invention relates to a method to specifically stain collagen producing cells in tissue sections and to immunhistologically diagnose fibrotic diseases using antibodies against human C-terminal procollagen α₁ (III) propeptide (PIIICP) and/or structurally related or homologous sequences.

### DESCRIPTION OF THE INVENTION

In this description a number of documents are cited. The disclosed content of these documents, including instructions of the manufacturer, is hereby incorporated by reference.

The invention comprises the use of antibodies directed against amino acid sequences (as antigens) that are homologous to parts of the amino acid sequence of C-terminal procollagen a, (III) propeptide (PIIICP) and/or are structurally related. Therefore non biotechnologically produced compounds are also comprised. The nucleotide sequence of human PIIICP has been deposited in the Genebank (Accession No. X14420 and X01742). The amino acid sequence of this peptide is shown in FIG. 1 (I) as an example. The propeptide sequence is indicated in the appendix in the context of the whole procollagen sequence C-terminal of the procollagen C proteinase cleavage site.

Especially preferred is the use of monoclonal and/or polyclonal antibodies that are characterized by the fact that they bind to an epitope in the C-terminal region of PIIICP.

Especially preferred is the use of the monoclonal antibody 48D19 (Burchardt E R, Heke M, Kauschke S G, Harjes P, Kohlmeyer J, Kroll W, Schauer M, Schroeder W, Voelker M. Epitope-specific monoclonal antibodies against human C-terminal procollagen alpha1(III)-propeptide. Matrix Biol 1998 December;17(8-9):673-7) that is characterized by the fact that it binds to an epitope within the C-terminal region of PIIICP.

The invention can be used as a qualitative and/or quantitative method to visualize fibrogenesis in histological tissue sections by the selective staining of collagen producing cells.

Preferred is the use of the invention as a qualitative and/or quantitative method to visualize fibrogenesis in histological tissue sections of the liver by the selective staining of hepatic stellate cells.

By contrast, methods that are limited to staining total collagen with Sirius Red/Fast Green only measure the total collagen deposition but not the current collagen production of collagen producing cells.

α-smooth muscle cell actin (α-SMA) is by contrast only an indirect marker of transformation, from which scar formation cannot be directly inferred.

Furthermore, the invention can be used to monitor therapies with antifibrotic compounds.

Preferred is the use of the invention as a method to monitor the therapy with recombination PIIICP.

Commonly, it has been shown to be of advantage to use the concerned antibody or antibodies at a concentration of 5 μg/ml for the incubation with the histological sections.

Under some circumstances it be of advantage, however, to deviate from the above specified amounts or concentrations, respectively, depending on the nature of the histological sections and the content of PIIICP antigen.

### BACKGROUND OF THE INVENTION

Collagen Biosynthesis

Collagens of types I and III are synthesized as prepropeptides and are extensively modified posttranslationally. Among the intracellular modifications are glycosylations, enzymatic hydroxylation reactions involving lysine and proline in its 3- and 4-positions. The modified propeptides spontaneously assemble into [α₁(III)]₃ homotrimers in the case of collagen (III). In the case of collagen type I mostly [α₁(I)]₂α₂(I) heterotrimers as well as—to a lesser extent—[α₁(I)]₃ homotrimers are formed. After exocytosis, the propeptides are first cleaved at the C-terminus of the nascent collagen and then at the N-terminus by a set of specific endoproteases. The cleavage resulting in the C-terminal procollagen propeptide (PIIICP) is catalyzed by the procollagen C-proteinase, an astacin-like metalloprotease, which is identical to the bone morphogenetic protein-1. Different tissue-specific expression patterns of different splice variants of the BMP-1 protein have been discovered. Furthermore, additional enzymes that are homologous to BMP-1 have been discovered, that are responsible for the cleavage of-PIIICP, such as mTLL-1 (mammalian tolloid-like 1) and the tolloid from Drosophila.

The N-terminal procollagen propeptide of collagen I (PINP) is cleaved off by the same N-proteinase that also digests the N-terminal procollagen propeptide of collagen type II. By contrast, N-terminal procollagen (III) propeptide (PIIINP) is cleaved by off by a proteinase activity distinct from the N-proteinase (I and II). The responsible enzyme is called procollagen N-proteinase type III and is part of a family of extracellular proteases, termed ADAMTS (a disintegrin and metalloprotease with thrombospondin motifs.

PIIICP

Human C-terminal Procollagen α₁ (III) propeptide (PIIICP) is a proteolytic fragment, emanating from the cleavage of procollagen α₁ (III) (PIIIP) by procollagen-C-proteinase (PCP). PIIIP is the characteristic collagen of parenchymatous organs.

PIIICP occurs as a trimer consisting of three identical monomeric PIIICP subunits that are linked by intermolecular disulfide bridges. Theoretical structural considerations and site-directed mutagenesis experiments with so-called collagen mini genes have led to the conclusion that at least 4 and probably even 6 cysteine residues of each monomeric PIIICP subunit are involved in intramolecular disulfide bridge formation. It is likely that only the cysteine residues in positions 51 and 68 are involved in intermolecular disulfide bridge formation.

So far only methods for the quantification of PIIICP in liquids have been described (WO09924835A2 und EPO0988964A1). In the patent application a specific staining of hepatic stellate cells using monoclonal antibodies against PIIICP is reported. Burchardt et. al., 1998, see above) refer to a possible application of antibodies against PIIICP for immunohistological studies. Utterly surprisingly it has now been discovered that collagen producing cells are selectively stained using specific antibodies against human PIIICP in histological tissue sections from human liver and that, in this way, collagen production can be determined qualitatively and/or quantitatively.

Fibrotic Diseases

Fibrotic diseases are defined as a diverse group of diseases that are associated with a qualitatively altered collagen production or with an increased deposition of collagen in the exrtracellular space. To this group of diseases belong, among others, systemic or localized scleroderma, liver fibrosis of various etiologies, alcoholic cirrhosis, e.g. alcoholic liver cirrhosis, biliary cirrhosis, hepatitis of viral or other origin, veno-occlusive disease, idiopathic interstitial fibrosis, idiopathic pulmonary fibrosis, interstitial pulmonary fibrosis, acute pulmonary fibrosis, acute respiratory distress syndrome, perimuscular fibrosis, pericentral fibrosis, dermatofibroma, kidney fibrosis, diabetic nephropathy, glomerulonephritis, keloids, hypertrophic scar formation, joint adhesions, arthrosis, myelofibrosis, corneal scaring, cystic fibrosis, muscular fibrosis, Duchenne's muscular dystrophy, esophageal stricture, retroabdominal scaring, Crohn's disease, ulcerative colitis, atherosclerotic alterations, pulmonary hypertension, angiopathy of the arteries and veins, aneurysms of large vessels.

Further fibrotic diseases are induced or initiated by scar revisions, plastic surgeries, glaucoma, cataract fibrosis, corneal scaring, graft vs. host disease, tendon surgery, nerve entrapment, Dupuytren's contracture, OB/GYN adhesions, pelvic adhesions, infertility, peridural fibrosis, diseases of the thyroid gland or the parathyroids, metastatic bone disease, multiple myeloma, or restenoses.

Liver Fibrosis

Liver fibrosis is characterized by an increased production of extracellular matrix components which form hepatic scars. The extracellular matrix primarily consists of fibril-forming collagens, particularly collagens of type I and III, matrix-glycoconjugates such as proteoglycanes, fibronectins and hyaluronic acid. The main producers of the extracellular matrix are activated hepatic stellate cells. In the healthy liver, hepatic stellate cells are quiescent cells that store retinoids and only produce small amounts of extracellular matrix proteins. By contact with fibrogenic stimuli hepatic stellate cells assume an activated phenotype which is characterized by a loss of the stored retinoid, by increased proliferation and by a morphological resemblance to myofibroblasts. In addition, activated hepatic stellate cells present with an increased expression of new genes, such as α-smooth muscle actin (α-SMA), ICAM-1, chemokines und cytokines.

Fibrillary collagens of types I und III are the main expression products of activated hepatic stellate cells. In the course of collagen synthesis, C-terminal procollagen α₁(III) propeptide (PIIICP) is formed by the cleavage of procollagen α₁ (III) by procollagen C-proteinase.

More recent results indicate that the cellular basis of liver fibrosis is the activation of hepatic stellate cells by fibrotic stimuli. The hepatic stellate cell changes its phenotype, proliferates und start to synthesize extracellular matrix proteins. If the rate of extracellular matrix protein synthesis exceeds the rate of collagen degradation a fibrotic scar is formed. When the fibrotic stimulus ceases to exist and the rate of collagen degradation exceeds the rate of collagen deposition, the fibrotic scar material is degraded. The resolution of fibrillary collagen—and concommittantly of the scar structure—is accompanied by an apoptosis of activated hepatic stellate cells und therefore by the disappearance of the cellular basis of fibrosis.

By the selective staining of hepatic stellate cells and the quantification of their collagen production it becomes possible to investigate specific changes with regard to fibrogenesis and to directly determine the status of collagen synthesis of hepatic stellate cells.

Immunohistochemical methods comprising PIIICP as the antigen can be used as surrogate markers for the measurement and monitoring of the disease progress of liver fibrosis.

### EXAMPLES

### Example 1

Application of an Immunohistological Method for the Diagnosis of the Antifibrotic Effect of Lamivudine in the Liver

Paired liver tissue samples were obtained from 80 patients with chronic hepatitis B. The patients participated in two phase III clinical trials in multicenter studies in North America. The study was carried out according to a protocol that had received approval from the Human Investigations Committee of the University of North Carolina at Chapel Hill.

After randomization, the concerned patients were either treated with a daily dose of 100 mg lamivudine or with placebo for 52 weeks. A liver tissue sample was obtained before the start and at the end of the therapy. The liver tissue samples were examined by a pathologist. The comparison and scoring of the tissue sections were carried out in a blinded fashion using the histology activity index (HAI). A reduction of the HAI by 2 points HAI is considered a significant change in hepatic histology after the termination of the treatment. The used tissue sections for the immunhistochemical diagnostic procedures were selected according to the mode of treatment (lamivudine or placebo) and according to the location of origin (limited to North America). All available biopsy sections that had been carried out in a pair wise fashion in the two studies and that fell under the above mentioned criteria, were included in the analysis.

### Example 2

Immunohistochemical Detection of α-SMA and PIIICP

Paraffin-embedded tissue sections were stained for α-smooth muscle actin (α-SMA) or PIIICP (FIG. 2). The staining procedure was carried out using the DAKO Envision System (DAKO; Carpinteria, Calif., USA). After inhibiting endogenous peroxidase with Peroxidase Blocking Agent (DAKO) the tissue sections were incubated for 10 minutes at room temperature with a murine monoclonal anti-α-SMA antibody (diluted 1:200 in PBS+1% BSA) or with a murine monoclonal anti-PIIICP antibody (antibody concentration 5 mg/ml; diluted 1:100 in PBS+1% BSA). After the incubation, the sections were washed twice with PBS buffer for 3 minutes, respectively. The tissue sections were then incubated with labelled polymer (peroxidase-labelled polymer conjugated with anti-rabbit and anti-mouse immunoglobulins from the goat) at room temperature for 10 minutes. After washing twice with PBS buffer the tissue sections were incubated with 3,3-diaminobenzidine (DAB) substrate for 8 minutes, rinsed with water and exposed to DAB Enhancer (Innovex Bioscience; Richmond, Calif., USA) for 5 minutes and subsequently rinsed with water again. Counterstaining was performed with hematoxyline. As a negative control all samples were incubated with 1% BSA solution instead of antibody.

### Example 3

Computerized Image and Data Analysis of α-SMA and PIIICP Stains

The stained parenchymal areas on the tissue sections, that are formed by α-SMA or PIIICP-positive hepatic stellate cells, were quantified with the Bioquant TCW 98 image analysis program (Bioquant Image Analysis; www.bioquant.com) with the connected Computer as well as with an Olympus microscope. The results were stated as the percent ratio between the stained area and the total area.

Tissue sections were analyzed in a blindfolded fashion without knowledge of the treatment method (placebo or lamivudine).

The baselines and the quantitative immunostains against α-SMA und PIIICP were compared between lamivudine und placebo patients using Student's t-tests. In addition, the results of the immunostains were correlated with the loss of the hepatitis B e-antigen (HBeAg) using the Mantel-Haenszel chi square test.

The immunohistochemical quantification was futhermore correlated with changes of individual components of the histological activity index (HAI) or with entire HAI. For the individual patients in both treatment groups—lamivudine or placebo, respectively—the change of α-SMA- or PIIICP-stained areas was calculated according to: ΔSMA (or ΔPIIICP)=percent area before treatment minus percent treatment after treatment. The mean change was compared between the lamivudine-treated and the placebo-treated groups using Student's t-Test.

### Example 4

Quantification of α-SMA and PIIICP

The serological und histological parameters of the patients before and after the therapy with lamivudine oder placebo are listed in table 1. The mean patient age in both patient groups was similar (42±12.6 years for the lamivudine group; 43±12.1 years for the placebo group), as well as the sex distribution (89% male (lamivudine) versus 82% male (placebo)). Patients in the lamivudine group had lower concentrations of the hepatitis B virus (HBV) DNA as well as a reduced lobular necrosis after the treatment compared to before the treatment. Fibrosis scores were 2.1±1.2 versus 1.8±1.2 in the lamivudine group (before and after therapy, p=0.06) and 2.1±1.3 versus 2.3±1.4 in the placebo group (before and after therapy, p=0.19). In addition, patients treated with lamivudine lost the HbeAg more often than patients treated with placebo (36.2% versus 9%; p<0.05).

TABLE 1
Serological and histological parameters of patients with chronic hepatitis
B before and after treatment with lamivudine or placebo.

| | Lamivudine (n = 47) | | Placebo (n = 33) | |
|---|---|---|---|---|
| | Pre | Post | Pre | Post |
| ALT^{a} | 154 ± 100 | 60.7 ± 55** | 155 ± 120 | 103.6 ± 109* |
| (IU/l, | | | | |
| mean ± SD^{b}) | | | | |
| HBV^{c} DNA | 180 ± 211^{ψ} | 33 ± 120** | 110 ± 113 | 70.7 ± 111* |
| (pg/ml) | | | | |
| HAI^{d}-score | | | | |
| (mean ± | | | | |
| SD^{b}) | | | | |
| Periportal | 3.2 ± 1.2 | 2.2 ± 1.2** | 3.2 ± 0.2 | 2.9 ± 0.9 |
| inflammation | | | | |
| Lobular | 2.0 ± 1.2^{ψ} | 1.3 ± 1.0** | 2.7 ± 1.2 | 1.8 ± 1.2** |
| Necrosis | | | | |
| Portal | 2.7 ± 1.0 | 2.1 ± 1.0** | 2.9 ± 0.5 | 2.7 ± 0.8 |
| inflammation | | | | |
| Fibrosis | 2.1 ± 1.2 | 1.8 ± 1.2 | 2.1 ± 1.3 | 2.3 ± 1.4 |
| Total Kno- | 10.0 ± 3.6 | 7.4 ± 3.1** | 10.9 ± 0.5 | 9.8 ± 2.8* |
| dell scoret | | | | |

^{a}Alanine amino transferase;
^{b}Standard deviation;
^{c}Hepatitis B virus;
^{d}Histological activity index
*p = 0.05, **p < 0.01, Pre- vs. posttreatment with either lamivudine or placebo.
^{ψ}p < 0.05 pretreatment lamivudine group vs. placebo group.

The α-SMA values were increased in patients with a higher degree of fibrosis. For patients with fibrosis scores of 3 or 4 the mean α-SMA value was 1.46±0.23 compared to 0.53±0.07 for patients with fibrosis scores of 0 or 1 (p<0.05). Liver tissue samples from patients treated with lamivudine exhibited a significantly lower α-SMA expression (1.06±0.23 versus 0.58±0.11—before and after treatment—p<0.05). By contrast, the α-SMA values of patients treated with placebo were increased (0.82±0.14 versus 1.32±0.21; before vs. after treatment; p<0.05).

The overall expression levels of PIIICP were lower than those for α-SMA. Expression values of PIIICP were reduced to a degree comparable to α-SMA after treatment with lamivudine in comparison to placebo treatment (0.06 (before lamivudine), 0.02 (after lamivudine), p<0.05; and 0.06 (before Placebo), 0.05 (after Placebo); p not significant).

According to the current state of the art, α-SMA is produced by activated hepatic stellate cells and is used as a marker of transformation from the quiescent to the activated hepatic stellate cell type in immunhistological tissue sections.

### Example 5

Changes in α-SMA and PIIICP Expression in the Lamivudine or Placebo Groups in Correlation with the Concentration of Serum Alanine Amino Transferase

The changes in the serum concentration of alanine mino transferase (ALT) were correlated with the expression of α-SMA and PIIICP (table 2). Patients treated with lamivudine who showed a decreased serum ALT activity exhibited a decreased expression of α-SMA and PIIICP. In lamivudine-treated patients with an increased serum ALT activity the expression of α-SMA and PIIICP was increased, however. By contrast, placebo-treated patients did not show a significant change of α-SMA and PIIICP expression, independent of the serum ALT activity.

TABLE 2
Percentage ratio of the α-SMA- or PIIICP-stained area, respectively,
over the total area before and after treatment with lamivudine or
placebo stratified according to serum-ALT activity (n =
lamivudine/placebo).

| Serum | Lamivudine (n = 47) | | | | Placebo (n = 33) | | | |
|---|---|---|---|---|---|---|---|---|
| ALT^{a}- | α-SMA | | PIIIC | | α-SMA | | PIIICP | |
| Activity | Pre | Post | Pre | Post | Pre | Post | Pre | Post |
| Increased | 1.18 | 0.56** | 0.07 | 0.02* | 0.92 | 1.44^{ψ} | 0.02 | 0.06 |
| (n = 39/ | | | | | | | | |
| 20) | | | | | | | | |
| Decreased | 0.47 | 0.65 | 0.01 | 0.04 | 0.66 | 1.15^{ψ} | 0.12 | 0.05 |
| (n = 8/13) | | | | | | | | |

^{a}Alanine amino transferase
**p < 0.01,
*p < 0.05,
^{ψ}p = 0.06

serum-ALT activity (n=lamivudine/placebo).

### Example 6

Changes of the α-SMA and PIIICP Expression in the Lamivudine and Placebo Groups in Correlation with the Histological Activity Indexes (HAI, Necroinflammatory Score, Fibrosis Score)

For liver tissue samples with improved necroinflammatory scores, α-SMA and PIIICP expression in the lamivudine-treated samples were reduced. For lamivudine-treated patients with unchanged necroinflammatory scores the α-SMA expression was reduced while the PIIICP expression remained unchanged (table 3). In lamivudine-treated patients with worsened necroinflammatory scores the α-SMA expression and the PIIICP expression were increased after the treatment versus before initiation of the therapy. Placebo-treated patients showed an increased α-SMA and PIIICP expression in the liver tissue samples, independently of the fibrosis score.

TABLE 3
Change of α-SMA and PIIICP before and after treatment with lamivudine or
placebo stratified according to the histological activity index (HAI, n = lamivudine/
placebo) score. The expression of α-SMA or PIIICP, respectively, was quantified as
percentage ratio of the positively stained area over the total area.

| | Lamivudine (n = 47) | | | | Placebo (n = 33) | | | |
|---|---|---|---|---|---|---|---|---|
| | α-SMA | | PIIICP | | α-SMA | | PIIICP | |
| | Pre | Post | Pre | Post | Pre | Post | Pre | Post |
| Necroinflammatory | | | | | | | | |
| score | | | | | | | | |
| Improved (n = 33/12) | 1.30 | 0.60** | 0.08 | 0.02* | 1.00 | 1.50 | 0.15 | 0.17 |
| Unchanged (n = 7/19) | 0.50 | 0.20 | 0.01 | 0.01 | 0.73 | 1.30* | 0.03 | 0.80* |
| Worsened (n = 7/2) | 0.50 | 0.80 | 0.01 | 0.04 | 0.80 | 1.10 | 0.01 | 0.01 |
| Fibrosis score | | | | | | | | |
| Improved (n = 10/4) | 1.02 | 0.23** | 0.03 | 0.01* | 1.74 | 1.30 | 0.01 | 0.05 |
| Unchanged (n = 31/21) | 1.01 | 0.65^{ψ} | 0.08 | 0.02 | 0.73 | 1.20 | 0.03 | 0.06 |
| Worsened (n = 6/8) | 1.35 | 0.77 | 0.02 | 0.04 | 0.62 | 1.57 | 0.20 | 0.04 |

**p < 0.01,
*p < 0.05,
^{ψ}p = 0.06

In lamivudine-treated patients with improved fibrosis scores the α-SMA and PIIICP expression was decreased in the liver tissue sections after the treatment. For lamivudine-treated patients with unchanged fibrosis scores the α-SMA and PIIICP expression were also decreased in the liver tissue sections after treatment.

However, the α-SMA expression was decreased in lamivudine-treated patients with worsened fibrosis scores in the liver tissue sections after the treatment, while the PIIICP expression was increased.

In placebo-treated patients with improved or unchanged fibrosis scores, respectively, the α-SMA and PIIICP expression were decreased in the liver tissue sections after the treatment. For placebo-treated patients with worsened fibrosis scores the α-SMA expression was decreased in the liver tissue sections and the PIIICP expression was increased after the treatment.

Among all patients with improved or unchanged fibrosis scores the PIIICP expression was significantly decreased after lamivudine treatment, while it was significantly increased after placebo treatment.

α-SMA expression levels exhibited marked differences compared to the expression of PIIICP in the cases considered here, which could point to different roles of these molecules in liver fibrosis.

### Example 7

Change of the Fibrosis Score, of the α-SMA- and the PIIICP Expression in Lamivudine-Treated Groups in Correlation to the Loss of the Hepatitis B e Antigen (HbeAg)

In patients treated with lamivudine the loss of the HbeAg was correlated with improved fibrosis scores and decreased α-SMA and PIIICP expression in comparison with patients with residual HbeAg (table 4).

TABLE 4
Change of the fibrosis index, of the α-SMA-and PIIICP expression of
lamivudine-treated patients stratified according to HbeAg status.

| | | | No loss | |
|---|---|---|---|---|
| | HbeAg loss (n = 17) | | of HbeAg (n = 30) | |
| | Pre | Post | Pre | Post |
| Fibrosis | 2.35 | 1.65** | 1.90 | 1.93 |
| score | | | | |
| α-SMA | 1.46 | 0.54* | 0.83 | 0.59 |
| PIIICP | 0.10 | 0.02 | 0.03 | 0.03 |

**p < 0.01; *p < 0.05

In lamivudine-treated patients with residual HbeAg the fibrosis score and the PIIICP expression remained unchanged, while the α-SMA expression was decreased after the treatment. Clinical studies that investigate histological changes in liver tissue before and after a treatment require methods to enable the qualitative and/or quantitative measurement of such changes. An exact definition of an optimal scoring system as well as the prevention of interobserver variability are still debated. A change of at least two points in the histological index (HAI) is nevertheless considered significant and reproducible. The largest significant changes in hepatic histological investigations are observed in the reduction of necroinflammatory activity of patients who respond successfully to a treatment. Measurable improvements in the course of liver fibrosis are only observed to a minor extent.

Due to the short length of most antiviral treatment protocols it is possible that the potential effects of the treatment on the course of fibrosis are underestimated and that subtle changes in fibrotic depositions are not detected.

By the use of an immunhistochemical method using antibodies directed against PIIICP it is now possible to directly detect early steps in the course of liver fibrosis.

The most marked reduction of PIIICP expression was found in patients with improved or unchanged fibrosis scores after lamivudine treatment. By contrast the PIIICP expression was increased when the fibrosis score worsened under the lamivudine treatment. In placebo-treated patients the expression was increased regardless of the fibrosis score. This suggests an advantageous effect of the lamivudine therapy on the course of fibrogenesis.

The data presented here suggest that PIIICP, as an immunohistochemical marker, is a sensitive method of detection for changes in fibrogenesis or ongoing collagen production, respectively. By contrast, conventional total collagen stains do not measure ongoing collagen production and are therefore not very sensitve in the detection of changes within shorter timeframes.

The use of α-SMA as an immunohistochemical marker of fibrogenesis yields similar results. However, while the expression of PIIICP is increased when the fibrosis score worsens in the course of lamivudine treatment, the expression of α-SMA-Expression decreases, pointing to a more indirect correlation between the fibrosis and changes in α-SMA expression compared to PIIICP.

### DESCRIPTION OF THE FIGURES

FIG. 1 shows the amino acid sequence in the vicinity of the human C-terminal procollagen α₁(III) propeptide (PIIICP). The subsequence encompassing the binding epitope of the monoclonal antibody 48D19 is underlined.

FIG. 2 shows a histological section of fibrotic livers from studied patients. The concerned images show negative controls in which the section was incubated with a 1% (w/v) BSA solution instead of anti-PIIICP antibody solution. A specific staining of cells is not discernable.

FIGS. 3 to 5 show histological sections of fibrotic livers from studied patients. The sections were incubated with anti-PIIICP antibody solution. A specific staining of collagen-producing cells is discernable.

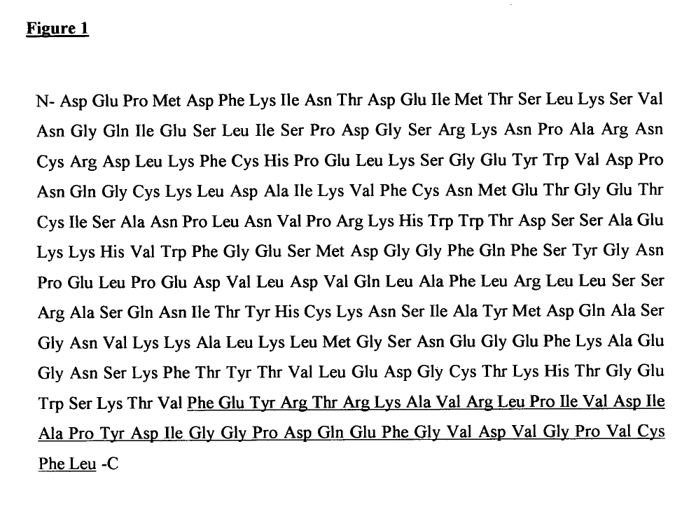

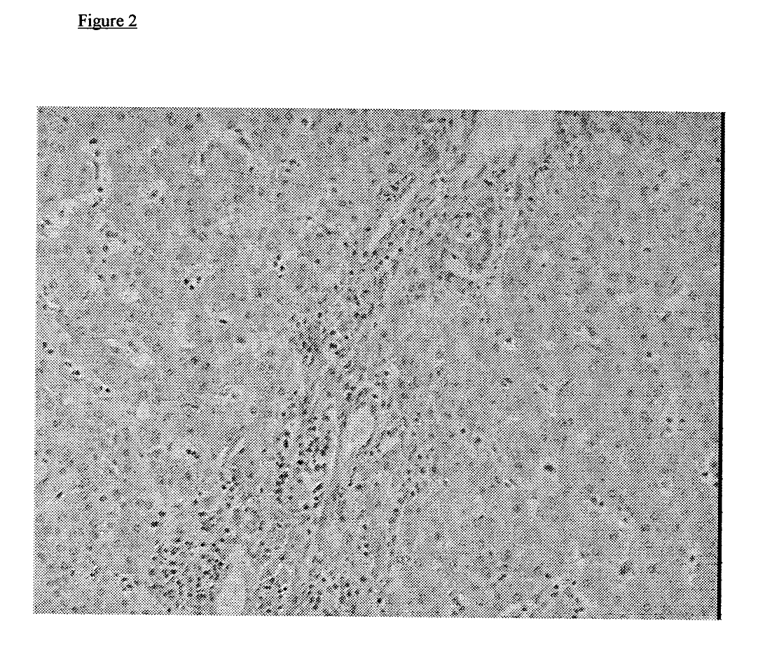

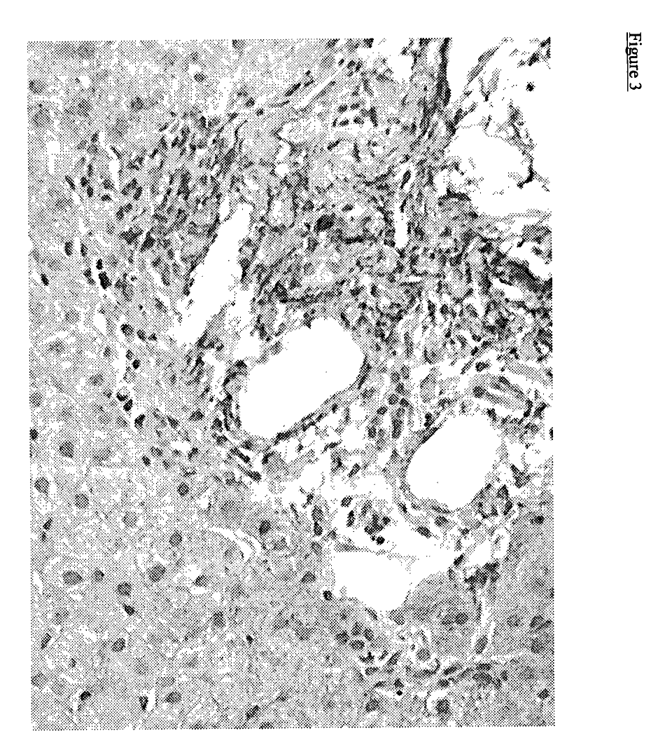

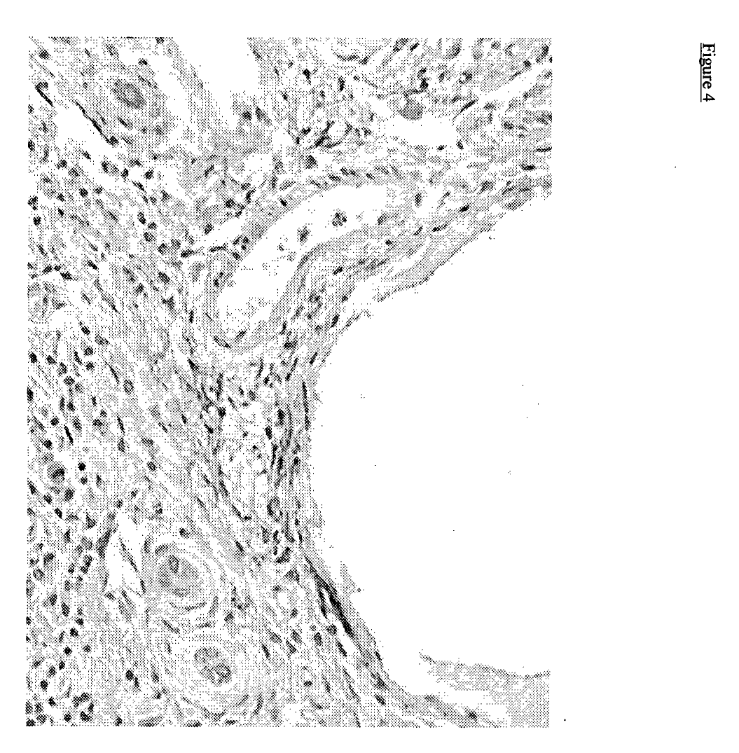

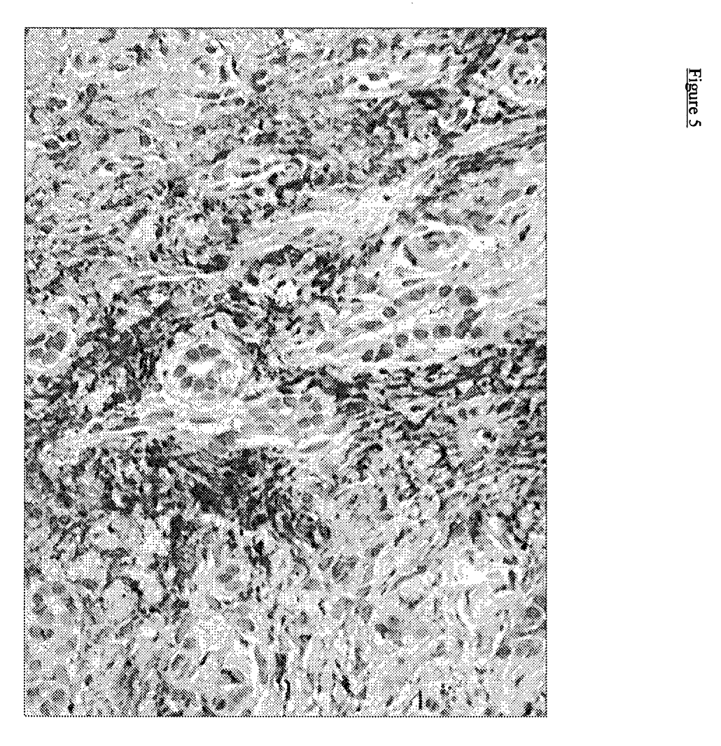

1. An immunohistological method to detect fibrogenesis in histological tissue sections comprising:
(a) antibodies directed against the C-terminal procollagen α₁ (III) propeptide and/or structurally related or homologous sequences
(b) bringing said antibodies into contact with the sample containing the antigen
(c) detecting the formed antigen antibody complex
(d) determining the stained area qualitatively and/or quantitatively,
whereby the progression of fibrotic diseases can be predicted.

2. An immunohistochemical method according to claim 1 wherein fibrogenesis is visualized in histological sections of human tissues.

3. An immunohistochemical method according to claim 2 wherein fibrogenesis is visualized in histological sections of human livers.

4. An immunohistochemical method according to claim 1 wherein said method is used to diagnose and/or diagnostically monitor the course of fibrotic diseases.

5. An immunohistochemical method according to claim 1 wherein said method is used to monitor antifibrotic therapies.

6. An immunohistochemical method according to claim 1 wherein said method is used to monitor therapies with recombinant C-terminal procollagen α₁(III) propeptide.

7. An immunohistochemical method according to claim 1 wherein said monoclonal and/or polyclonal antibodies recognize an epitope within the more C-terminal part of C-terminal procollagen α₁(III) propeptide.

8. An immunohistochemical method according to claim 1 wherein said antibody is mAb 48D19.

9. An immunohistochemical method according to claim 1 wherein the detection of said antigen-antibody complex is carried out by directly labelling the directed antibody radioactively, by conjugation with an enzyme or a fluorgenic probe and/or with the aid of a secondary antibody.
